(19) United States
(12) Patent Application Publication
(10) Pub. No.: US 2009/0226971 A1
(43) Pub. Date: Sep. 10, 2009

(54) Portable Rapid Microfluidic Thermal Cycler for Extremely Fast Nucleic Acid Amplification

(76) Inventors: Neil Reginald Beer, Pleasanton, CA (US); Kambiz Vafai, Mission Viejo, CA (US); Shadi Mahjoob, Riverdale, CA (US)

Correspondence Address: Lawrence Livermore National Security, LLC, LAWRENCE LIVERMORE NATIONAL LABORATORY, PO BOX 808, L-703, LIVERMORE, CA, 94551-0808, US

(21) Appl. No.: 12/270,030

(22) Filed: Nov. 13, 2008

Related U.S. Application Data
(60) Provisional application No. 61/022,692, filed on Jan. 22, 2008.

(51) Int. Cl. C12P 19/34; C12M 1/00

(52) U.S. Cl. 435/91.2; 435/303.1

(57) ABSTRACT

A portable apparatus for thermal cycling a material to be thermal cycled includes a portable microfluidic-compatible platform, a microfluidic heat exchanger carried by the portable microfluidic-compatible platform; a porous medium in the microfluidic heat exchanger; a microfluidic thermal cycling chamber containing the material to be thermal cycled, the microfluidic thermal cycling chamber operatively connected to the microfluidic heat exchanger; a working fluid at first temperature, a first system for transmitting the working fluid at first temperature to the microfluidic heat exchanger; a working fluid at a second temperature, a second system for transmitting the working fluid at second temperature to the microfluidic heat exchanger; a pump for flowing the working fluid at the first temperature from the first system to the microfluidic heat exchanger and through the porous medium; and flowing the working fluid at the second temperature from the second system to the heat exchanger and through the porous medium.

### CROSS-REFERENCE TO RELATED APPLICATIONS

The present application claims benefit under 35 U.S.C. § 119(e) of U.S. Provisional Patent Application No. 61/022,692 filed on Jan. 22, 2008 entitled “portable rapid microfluidic thermal cycler for extremely fast nucleic acid amplification,” the disclosure of which is hereby incorporated by reference in its entirety for all purposes. Related inventions are disclosed and claimed in U.S. patent application Ser. No. ______ titled Rapid Microfluidic Thermal Cycler for Nucleic Acid Amplification filed on the same as this application. The disclosure of U.S. patent application Ser. No. ______ titled Rapid Microfluidic Thermal Cycler for Nucleic Acid Amplification is hereby incorporated by reference.

### STATEMENT AS TO RIGHTS TO INVENTIONS MADE UNDER FEDERALLY SPONSORED RESEARCH AND DEVELOPMENT

The United States Government has rights in this invention pursuant to Contract No. DE-AC52-07NA27344 between the United States Department of Energy and Lawrence Livermore National Security, LLC for the operation of Lawrence Livermore National Laboratory.

### BACKGROUND OF THE INVENTION

1. Field of Endeavor

The present invention relates to thermal cycling and more particularly to a portable rapid microfluidic thermal cycler.

2. State of Technology

United States Published Patent No. 2005/0252773 for a thermal reaction device and method for using the same includes the following state of technology information:
“Devices with the ability to conduct nucleic acid amplifications would have diverse utilities. For example, such devices could be used as an analytical tool to determine whether a particular target nucleic acid of interest is present or absent in a sample. Thus, the devices could be utilized to test for the presence of particular pathogens (e.g., viruses, bacteria or fungi), and for identification purposes (e.g., paternity and forensic applications). Such devices could also be utilized to detect or characterize specific nucleic acids previously correlated with particular diseases or genetic disorders. When used as analytical tools, the devices could also be utilized to conduct genotyping analyses and gene expression analyses (e.g., differential gene expression studies). Alternatively, the devices can be used in a preparative fashion to amplify sufficient nucleic acid for further analysis such as sequencing of amplified product, cell-typing, DNA fingerprinting and the like. Amplified products can also be used in various genetic engineering applications, such as insertion into a vector that can then be used to transform cells for the production of a desired protein product.”

United States Published Patent No. 2008/0166793 by Neil Reginald Beer for sorting, amplification, detection, and identification of nucleic acid subsequences in a complex mixture provides the following state of technology information:
“A complex environmental or clinical sample 201 is prepared using known physical (ultracentrifugation, filtering, diffusion separation, electrophoresis, cytometry etc.), chemical (pH), and biological (selective enzymatic degradation) techniques to extract and separate target nucleic acids or intact individual particles 205 (e.g., virus particles) from background (i.e., intra- and extra-cellular RNA/DNA from host cells, pollen, dust, etc.). This sample, containing relatively purified nucleic acid or particles containing nucleic acids (e.g., viruses), can be split into multiple parallel channels and mixed with appropriate reagents required for reverse transcription and subsequent PCR (primers/probes/dNTPs/enzymes/buffer). Each of these mixes are then introduced into the system in such a way that statistically no more than a single RNA/DNA is present in any given microreactor. For example, a sample containing 106 target RNA/DNA would require millions of microreators to ensure single RNA/DNA distribution.
An amplifier 207 provides Nucleic Acid Amplification. This may be accomplished by the Polymerase Chain Reaction (PCR) process, an exponential process whereby the amount of target DNA is doubled through each reaction cycle utilizing a polymerase enzyme, excess nucleic acid bases, primers, catalysts (MgCl2), etc. The reaction is powered by cycling the temperature from an annealing temperature whereby the primers bind to single-stranded DNA (ssDNA) through an extension temperature whereby the polymerase extends from the primer, adding nucleic acid bases until the complement strand is complete, to the melt temperature whereby the newly-created double-stranded DNA (dsDNA) is denatured into 2 separate strands. Returning the reaction mixture to the annealing temperature causes the primers to attach to the exposed strands, and the next cycle begins.
The heat addition and subtraction powering the PCR chemistry on the amplifier device 207 is described by the relation:

Q=hA(T_{wall}−T_{∞})
The amplifier 207 amplifies the organisms 206. The-nucleic acids 208 have been released from the organisms 206 and the nucleic acids 208 are amplified using the amplifier 207. For example, the amplifier 207 can be a thermocycler. The nucleic acids 208 can be amplified in-line before arraying them. As amplification occurs, detection of fluorescence-labeled TaqMan type probes occurs if desired. Following amplification, the system does not need decontamination due to the isolation of the chemical reactants,”

U.S. Pat. No. 3,635,037 for a Peltier-effect heat pump provides the following state of technology information:
“The Peltier-effect has been used heretofore in heat pumps for the heating or cooling of areas and substances in which fluid-refrigeration cycles are disadvantageous. For example, for small lightweight refrigerators, compressors, evaporators and associated components of a vapor/liquid refrigerating cycle may be inconvenient and it has, therefore, been proposed to use the heat pump action of a Peltier pile. The Peltier effect may be described as a thermoelectric phenomenon whereby heat is generated or abstracted at the junction of dissimilar metals or other conductors upon application of an electric current. For the most part, a large number of junctions is required for a pronounced thermal effect and, consequently, the Peltier junctions form a pile or battery to which a source of electrical energy may be connected. The Peltier conductors and their junctions may lie in parallel or in series-parallel configurations and may have substantially any shape. For example, a Peltier battery or pile may be elongated or may form a planar or three-dimensional (cubic or cylindrical) array. When the Peltier effect is used in a heat pump, the Peltier battery or pile is associated with a heat sink or heat exchange jacket to which heat transfer is promoted, the heat exchanger being provided with ribs, channels or the like to facilitate heat transfer to or from the Peltier pile over a large surface area of high thermal conductivity. A jacket of aluminum or other metal of high thermal conductivity may serve for this purpose.”

### SUMMARY

Features and advantages of the present invention will become apparent from the following description. Applicants are providing this description, which includes drawings and examples of specific embodiments, to give a broad representation of the invention. Various changes and modifications within the spirit and scope of the invention will become apparent to those skilled in the art from this description and by practice of the invention. The scope of the invention is not intended to be limited to the particular forms disclosed and the invention covers all modifications, equivalents, and alternatives falling within the spirit and scope of the invention as defined by the claims.

The present invention provides a system for extremely fast continuous flow or batch PCR amplification of target nucleic acids in a compact, portable microfluidic-compatible platform. The present invention also provides a system for extremely fast thermal cycling, precise thermal control, and low power consumption due to innovative heat transfer characteristics. In addition, present invention also provides a method for thermally calibrating the system to ensure the proper heating and cooling set points are reached during the extremely rapid cycling.

In one embodiment the present invention provides a portable apparatus for thermal cycling a material to be thermal cycled, including a portable microfluidic-compatible platform, a microfluidic heat exchanger carried by the portable microfluidic-compatible platform; a porous medium in the microfluidic heat exchanger; a microfluidic thermal cycling chamber containing the material to be thermal cycled, the microfluidic thermal cycling chamber operatively connected to the microfluidic heat exchanger; a working fluid at first temperature, a first system for transmitting the working fluid at first temperature to the microfluidic heat exchanger; a working fluid at a second temperature, a second system for transmitting the working fluid at second temperature to the microfluidic heat exchanger; a pump for flowing the working fluid at the first temperature from the first system to the microfluidic heat exchanger and through the porous medium; and flowing the working fluid at the second temperature from the second system to the heat exchanger and through the porous medium.

In one embodiment the first system for transmitting the working fluid at first temperature to the microfluidic heat exchanger is a first container for containing the working fluid at first temperature and the second system for transmitting the working fluid at second temperature to the microfluidic heat exchanger is a second container for containing the working fluid at second temperature. In another embodiment the first system for transmitting the working fluid at first temperature to the microfluidic heat exchanger and the second system for transmitting the working fluid at second temperature to the microfluidic heat exchanger comprises a single container and separate line with a heater or cooler that are connected to provide the working fluid at first temperature to the microfluidic heat exchanger and to provide the working fluid at second temperature to the microfluidic heat exchanger.

In one embodiment the present invention provides a portable apparatus for thermal cycling a material to be thermal cycled. The apparatus includes a portable microfluidic-compatible platform, a microfluidic heat exchanger carried by the portable microfluidic-compatible platform; a porous medium in the microfluidic heat exchanger; a microfluidic thermal cycling chamber containing the material to be thermal cycled, the microfluidic thermal cycling chamber operatively connected to the microfluidic heat exchanger; a working fluid at first temperature, a first container for containing the working fluid at first temperature, a working fluid at a second temperature, a second container for containing the working fluid at second temperature, a pump for flowing the working fluid at the first temperature from the first container to the microfluidic heat exchanger and through the porous medium; and flowing the working fluid at the second temperature from the second container to the heat exchanger and through the porous medium. In another embodiment the present invention provides a method of thermal cycling a material to be thermal cycled between a number of different temperatures. The method includes the steps of providing a portable microfluidic-compatible platform, providing a microfluidic heat exchanger on the portable microfluidic-compatible platform, the microfluidic heat exchanger operatively positioned with respect to the material to be thermal cycled providing working fluid at a first temperature, flowing the working fluid at the first temperature to the microfluidic heat exchanger to hold the material to be thermal cycled at the first temperature, providing working fluid at a second temperature, and flowing the working fluid at the first temperature to the heat exchanger to hold the material to be thermal cycled at the second temperature.

The present invention has use in a number of applications. For example, the present invention has use in biowarfare detection applications. The present invention has use in identifying, detecting, and monitoring bio-threat agents that contain nucleic acid signatures, such as spores, bacteria, etc. The present invention has use in biomedical applications. The present invention has use in tracking, identifying, and monitoring outbreaks of infectious disease. The present invention has use in automated processing, amplification, and detection of host or microbial DNA in biological fluids for medical purposes. The present invention has use in genomic analysis, genomic testing, cancer detection, genetic fingerprinting. The present invention has use in forensic applications. The present invention has use in automated processing, amplification, and detection DNA in biological fluids for forensic purposes. The present invention has use in food and beverage safety. The present invention has use in automated food testing for bacterial or viral contamination. The present invention has use in environmental monitoring and remediation monitoring.

The invention is susceptible to modifications and alternative forms. Specific embodiments are shown by way of example. It is to be understood that the invention is not limited to the particular forms disclosed. The invention covers all modifications, equivalents, and alternatives falling within the spirit and scope of the invention as defined by the claims.

### BRIEF DESCRIPTION OF THE DRAWINGS

The accompanying drawings, which are incorporated into and constitute a part of the specification, illustrate specific embodiments of the invention and, together with the general description of the invention given above, and the detailed description of the specific embodiments, serve to explain the principles of the invention.

FIG. 1 illustrates one embodiment of the present invention.

FIG. 2 illustrates another embodiment of the present invention.

FIG. 3 illustrates yet another embodiment of the present invention.

FIG. 4 illustrates an embodiment of the present invention utilizing a glass micro array.

FIG. 5 illustrates an embodiment of the present invention utilizing microreactors.

FIG. 6 illustrates another embodiment of the present invention.

FIG. 7 illustrates yet another embodiment of the present invention.

### DETAILED DESCRIPTION OF SPECIFIC EMBODIMENTS

Referring to the drawings, to the following detailed description, and to incorporated materials, detailed information about the invention is provided including the description of specific embodiments. The detailed description serves to explain the principles of the invention. The invention is susceptible to modifications and alternative forms. The invention is not limited to the particular forms disclosed. The invention covers all modifications, equivalents, and alternatives falling within the spirit and scope of the invention as defined by the claims.

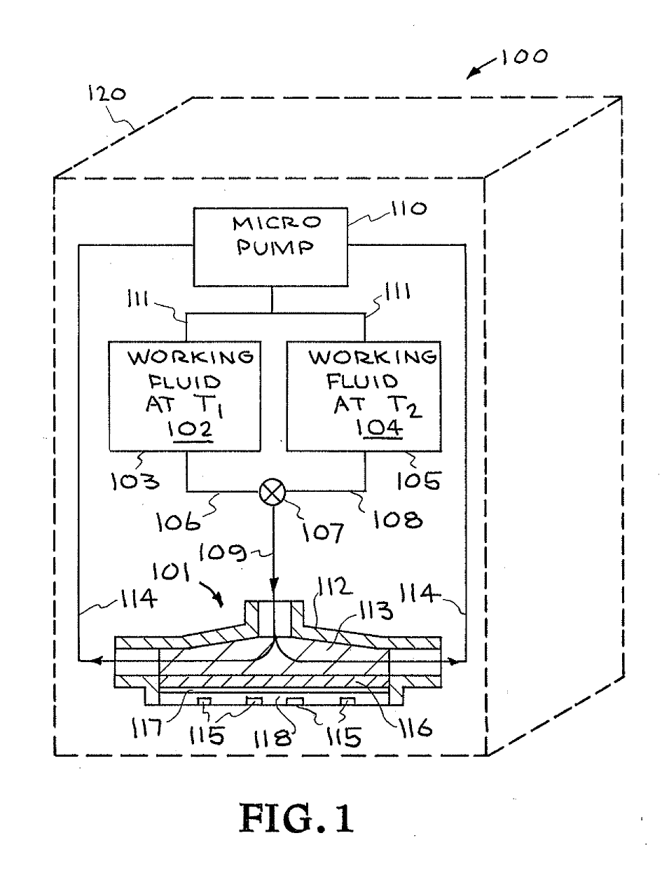

Referring now to the drawings and in particular to FIG. 1, one embodiment of a system constructed in accordance with the present invention is illustrated. The system is designated generally by the reference numeral 100. The system 100 provides extremely fast continuous flow or batch PCR amplification of target nucleic acids in a compact, portable microfluidic-compatible platform 120. Some of the technical challenges that were met in producing the system were (1) realizing a high throughput, field portable, real time PCR instrument that can run 10 assays in 1 minute, (2) a porous media heat exchanger coupled to an on-chip PCR device to optimize PCR (˜3 sec per cycle), and (3) field portable fluid reservoirs, valving, power supply, and pumps integrated with a real-time detector.

The system 100 provides thermal cycling a material 115 (DNA Sample) to be thermal cycled between a temperature T₁ and T₂ using a microfluidic heat exchanger 101 operatively positioned with respect to the material 115 to be thermal cycled. A working fluid 102 at T₁ is provided and the working fluid 102 at T₁ is flowed to the microfluidic heat exchanger 101. A working fluid 104 at T₂ is provided and the working fluid 104 at T₂ is flowed to the heat exchanger 101. The steps of flowing the working fluid at T₁ and at T₂ to the microfluidic heat exchanger 101 are repeated for a predetermined number of times. A porous medium 113 is located in the microfluidic heat exchanger 101. The working fluids at T₁ and T₂ flow through the porous medium 113 during the steps of flowing the working fluid at T₁ and T2 through the microfluidic heat exchanger 101. The system 100 is contained in a compact, portable microfluidic-compatible platform 120.

The material 115 to be thermal cycled is contained on a chip 118 (microarray 118) containing the DNA. Examples of microarrays are shown in U.S. Pat. No. 7,354,389 for a microarray detector and methods which states, “The present invention is directed to an analytic system for detection of a plurality of analytes that are bound to a biochip, wherein an optical detector uses registration markers illuminated by a first light source to determine a focal position for detection of the analytes that are illuminated by a second light source.” U.S. Pat. No. 7,354,389 for a microarray detector and methods is incorporated herein by reference. The DNA sample 115 is contained on the chip 118 containing the DNA sample. A highly conductive plate 116 connects the chip 118 to the heat exchanger 101. Conductive grease 117 is used to provide thermal conductivity between the chip 118 and the heat exchanger 101. Instead of conductive grease 117 between the chip 118 and the heat exchanger 101 other forms of connection may be used. For example, press-fit contact or thermally-conductive tape may be used between the chip 118 and the heat exchanger 101.

The steps of repeatedly flowing the working fluid at T₁ and at T₂ to the microfluidic heat exchanger 101 provide PCR fast and efficient nucleic acid analysis. The microfluidic polymerase chain reaction (PCR) thermal cycling method 100 is capable of extremely fast cycles, and a resulting extremely fast detection time for even long amplicons (amplified nucleic acids). The method 100 allows either singly or in combination: reagent and analyte mixing; cell, virion, or capsid lysing to release the target DNA if necessary; nucleic acid amplification through the polymerase chain reaction (PCR), and nucleic acid detection and characterization through optical or other means. An advantage of this system lies in its complete integration on a microfluidic platform and its extremely fast thermocycling.

The system 100 includes the following structural components: microfluidic heat exchanger 101, microfluidic heat exchanger housing 112, porous medium 113, micropump 110, lines 111, chamber 103, working fluid 102 at T₁, chamber 105, working fluid 104 at T₂, lines 106 and 108, multi position valve 107, line 109, highly conductive plate 116, thermal grease 117, chip containing DNA sample 118, and DNA sample 115.

The structural components of the system 100 having been described, the operation of the system 100 will be explained. The valve 107 is actuated to provide flow of working fluid 102 at T₁ from chamber 103 to the microfluidic heat exchanger 101, Micro pump 110 is actuated driving working fluid 102 at T₁ from chamber 103 to the microfluidic heat exchanger 101. The working fluid 102 at T₁ passes through the porous medium 113 in the microfluidic heat exchanger 101 raising the temperature of the material to be thermalcycled 115 to temperature T₁. The porous medium 113 in the microfluidic heat exchanger 101 results in substantial surface area enhancement and increased fluid flow-path tortuosity, both of which enhance heat transfer and the resulting heat flux between the working fluid and the porous matrix.

Next the valve 107 is actuated to provide flow of working fluid 104 at T₂ from chamber 105 to the microfluidic heat exchanger 101. Micro pump 110 is actuated driving working fluid 104 at T₂ from chamber 105 to the microfluidic heat exchanger 101. The working fluid 104 at T₂ passes through the porous medium 113 in the microfluidic heat exchanger 101 lowering the temperature of the material to be thermalcycled 115 to temperature T₂. The steps of flowing the working fluid at T₁ and at T₂ to the microfluidic heat exchanger 101 are repeated for a predetermined number of times to provide the desired PCR. The porous medium 113 in the microfluidic heat exchanger 101 results in substantial surface area enhancement and increased fluid flow-path tortuosity, both of which enhance heat transfer and the resulting heat flux between the working fluid and the porous matrix.

The heat exchanger 101 of the system 100 utilizes inlet and exit channels where heating/cooling fluid 102 and 104 is passing through, an enclosure, and a layer of conductive plate attached to a PCR micro-chip. The enclosure is filled with a conductive porous medium 113 of uniform porosity and permeability. In another embodiment the enclosure is filled with a conductive porous medium 113 with a gradient porosity. The nominal permeability and porosity of the porous matrix are taken as 3.74×10⁻¹⁰ m² and 0.45, respectively. The porous medium 113 is saturated with heating/cooling fluid 102, 104 coming through an inlet channel. The inlet channel will be connected to hot and cold supply tanks 103 and 105. A switching valve 107 is used to switch between hot 102 and cold tanks 105 for heating and cooling cycles. All lateral walls and top of the porous medium are insulated to minimize losses. The micropump 110 is positioned to drive the working fluids 102 and 105 directly into the microfluidic heat exchanger 101. By positioning the micropump 110 outside the hot and cold supply tanks 103 and 105 and lines to the microfluidic heat exchanger 101 it eliminates the time the would be required to bring the micropump 110 up to the new temperature after each change.

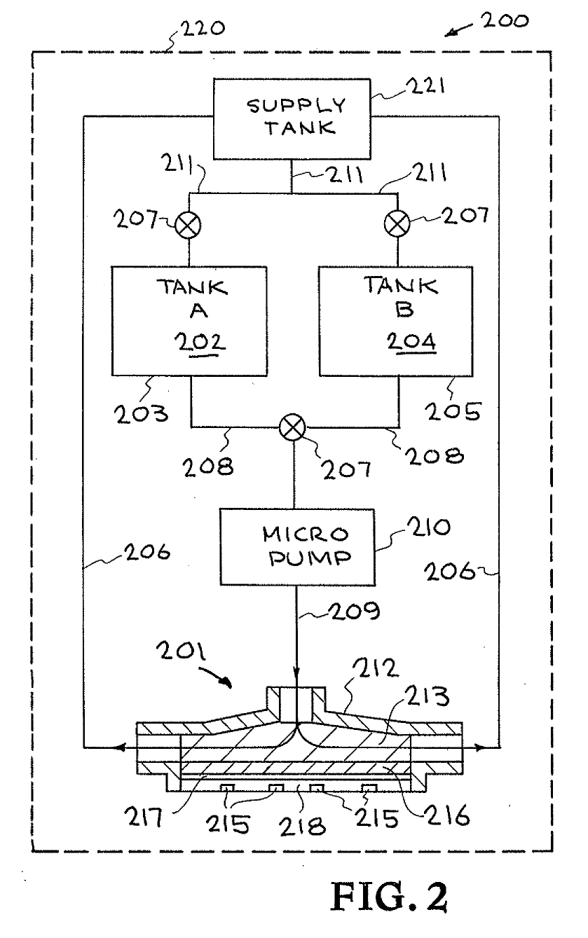

Referring now to FIG. 2, another embodiment of a system constructed in accordance with the present invention is illustrated. The system is designated generally by the reference numeral 200. The system 200 provides provides extremely fast continuous flow or batch PCR amplification of target nucleic acids in a compact, portable microfluidic-compatible platform 220. The material 215 to be thermal cycled is contained on a chip 218 (microarray 218) containing the DNA. The DNA sample 215 is contained on the chip 218 containing the DNA sample. A highly conductive plate 216 connects the chip 218 to the heat exchanger 201. Conductive grease is used to provide thermal conductivity between the chip 218 and the heat exchanger 201.

A working fluid 202 at T₁ is provided in “Tank A” 203. The working fluid is maintained at the temperature T₁ in Tank A (203) by appropriate heating and cooling equipment. The working fluid 202 at T₁ from Tank A (203) is flowed to the microfluidic heat exchanger 201.

A working fluid 204 at T₂ is provided in “Tank B” 205. The working fluid is maintained at the temperature T₂ in Tank B (205) by appropriate heating and cooling equipment. The working fluid 204 at T₂ from Tank B (205) is flowed to the heat exchanger 201.

The system 200 includes the following additional structural components: microfluidic heat exchanger housing 212, porous medium 213, lines 206, 208, 209, & 211, micropump 210, multiposition valves 207, and supply tank 221. The system 200 is contained in a compact, portable microfluidic-compatible platform 220.

The structural components of the system 200 having been described, the operation of the system 200 will be explained. When used for PCR, the system 200 provides thermal cycling a material 215 to be thermal cycled between a temperature T₁ and T₂ using a microfluidic heat exchanger 201 operatively positioned with respect to the material 215 to be thermal cycled. A working fluid 202 at T₁ is provided in “Tank A” 203. The working fluid 202 at T₁ from Tank A (203) is flowed to the microfluidic heat exchanger 201. A working fluid 204 at T₂ is provided in “Tank B” 205. The working fluid 204 at T₂ from Tank B (205) is flowed to the heat exchanger 201.

The multiposition valves 207 are actuated to provide flow of working fluid 202 at T₁ from Tank A (203) to the microfluidic heat exchanger 201. Micro pump 210 is actuated driving working fluid 202 at T₁ from Tank A (203) to the microfluidic heat exchanger 201. The working fluid 202 at T₁ passes through the porous medium 213 in the microfluidic heat exchanger 201 raising the temperature of the material to be thermalcycled 215 to temperature T₁. The porous medium 213 in the microfluidic heat exchanger 201 results in substantial surface area enhancement and increased fluid flow-path tortuosity, both of which enhance heat transfer and the resulting heat flux between the working fluid and the porous matrix.

Next the valves 207 are actuated to provide flow of working fluid 204 at T₂ from Tank B (205) to the microfluidic heat exchanger 201. Micro pump 210 is actuated driving working fluid 204 at T₂ from chamber 205 to the microfluidic heat exchanger 201. The working fluid 202 at T₂ passes through the porous medium 213 in the microfluidic heat exchanger 201 lowering the temperature of the material to be thermalcycled 215 to temperature T₂. The porous medium 213 in the microfluidic heat exchanger 201 results in substantial surface area enhancement and increased fluid flow-path tortuosity, both of which enhance heat transfer and the resulting heat flux between the working fluid and the porous matrix.

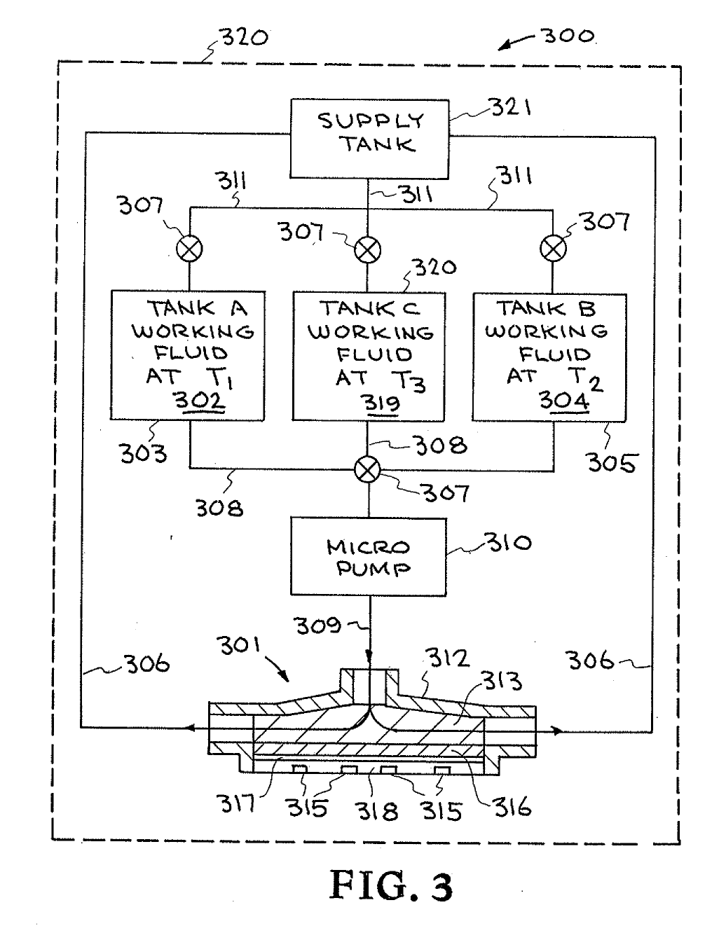

Referring now to FIG. 3, another embodiment of a thermal cycling system constructed in accordance with the present invention is illustrated. The system is designated generally by the reference numeral 300. The system 300 provides thermal cycling of a material 315 between different temperatures using a microfluidic heat exchanger 301 operatively positioned with respect to the material 315. The material to be thermal cycled 315 illustrated in FIG. 3 is a DNA sample. The DNA sample 315 is contained on the chip 318 containing the DNA sample. A highly conductive plate 316 connects the chip 318 to the heat exchanger 301. Conductive grease is used to provide thermal conductivity between the chip 318 and the heat exchanger 301.

A working fluid 302 at T₁ is provided in “Tank A” 303. The working fluid is maintained at the temperature T₁ in Tank A (303) by appropriate heating and cooling equipment. The working fluid 302 at T₁ from Tank A (303) is flowed to the microfluidic heat exchanger 301.

A working fluid 304 at T₂ is provided in “Tank B” 305. The working fluid is maintained at the temperature T₂ in Tank B (305) by appropriate heating and cooling equipment. The working fluid 304 at T₂ from Tank B (305) is flowed to the heat exchanger 301.

A working fluid 319 at T₃ is provided in “Tank C” 320. The working fluid is maintained at the temperature T₃ in Tank C (320) by appropriate heating and cooling equipment. The working fluid 319 at T₃ from Tank C (320) is flowed to the heat exchanger 301. The system 300 includes the following additional structural components: microfluidic heat exchanger housing 312, porous medium 313, lines 306, 308, 309, & 311, micropump 310, multiposition valves 307, and supply tank 321.

The structural components of the system 300 having been described, the operation of the system 300 will be explained. The system 300 will be described as a polymerase chain reaction (PCR) system; however, it is to be understood that the system 300 can be used as other thermal cycling systems.

When used for PCR, the system 300 provides thermal cycling a material 315 to be thermal cycled between a temperatures T₁ and T₂ and T₃ using a microfluidic heat exchanger 301 operatively positioned with respect to the material 315 to be thermal cycled. The material 315 to be thermal cycled is contained on a chip 318 (microarray 318) containing the DNA.

A working fluid 302 at T₁ is provided in “Tank A” 303. The working fluid 302 at T₁ from Tank A (303) is flowed to the microfluidic heat exchanger 301. A working fluid 403 at T₂ is provided in “Tank B” 305. The working fluid 303 at T₂ from Tank B (305) is flowed to the heat exchanger 301. A working fluid 319 at T₃ is provided in “Tank C” 320. The working fluid 319 at T₃ from Tank C (320) is flowed to the heat exchanger 301.

The multiposition valves 307 are actuated to provide flow of working fluid 302 at T₁ from Tank A (303) to the microfluidic heat exchanger 301. Micro pump 310 is actuated driving working fluid 302 at T₁ from Tank A (303) to the microfluidic heat exchanger 301. The working fluid 302 at T₁ passes through the porous medium 313 in the microfluidic heat exchanger 301 raising the temperature of the material to be thermalcycled 315 to temperature T₁. The porous medium 313 in the microfluidic heat exchanger 301 results in substantial surface area enhancement and increased fluid flow-path tortuosity, both of which enhance heat transfer and the resulting heat flux between the working fluid and the porous matrix.

Next the valves 307 are actuated to provide flow of working fluid 304 at T₂ from Tank B (305) to the microfluidic heat exchanger 301. Micro pump 310 is actuated driving working fluid 304 at T₂ from chamber 305 to the microfluidic heat exchanger 301. The working fluid 304 at T₂ passes through the porous medium 313 in the microfluidic heat exchanger 301 lowering the temperature of the material to be thermalcycled 315 to temperature T₂. The porous medium 313 in the microfluidic heat exchanger 301 results in substantial surface area enhancement and increased fluid flow-path tortuosity, both of which enhance heat transfer and the resulting heat flux between the working fluid and the porous matrix.

The valves 307 can also be actuated to provide flow of working fluid 319 at T₃ from Tank C (320) to the microfluidic heat exchanger 301. Micro pump 310 is actuated driving working fluid 319 at T₃ from Tank C (320) to the microfluidic heat exchanger 301. The working fluid 319 at T₃ passes through the porous medium 313 in the microfluidic heat exchanger 301 changing the temperature of the material to be thermalcycled 315 to temperature T₃. The porous medium 313 in the microfluidic heat exchanger 301 results in substantial surface area enhancement and increased fluid flow-path tortuosity, both of which enhance heat transfer and the resulting heat flux between the working fluid and the porous matrix.

The heat exchanger 301 of the system 300 utilizes inlet and exit channels where heating/cooling fluid 302, 304, and 319 pass through the porous media 313. In one embodiment the porous media 313 has a uniform porosity and permeability. The nominal permeability and porosity of the porous matrix are taken as 3.74×10⁻¹⁰ m² and 0.45, respectively. In other embodiments the porous media 313 has gradient porosity. The system 300 allows the heat exchanger 301 to change the temperature of the material to be thermal cycled 315 between and to a variety of different temperatures. By various combinations of settings of the multiposition valves 307 it is possible to supply working fluid from tanks A, B, and C at a near infinite variety of different temperatures. This provides a full spectrum of heat transfer control by a combination of T₁, T₂, and T₃ as well as coolant flow rate.

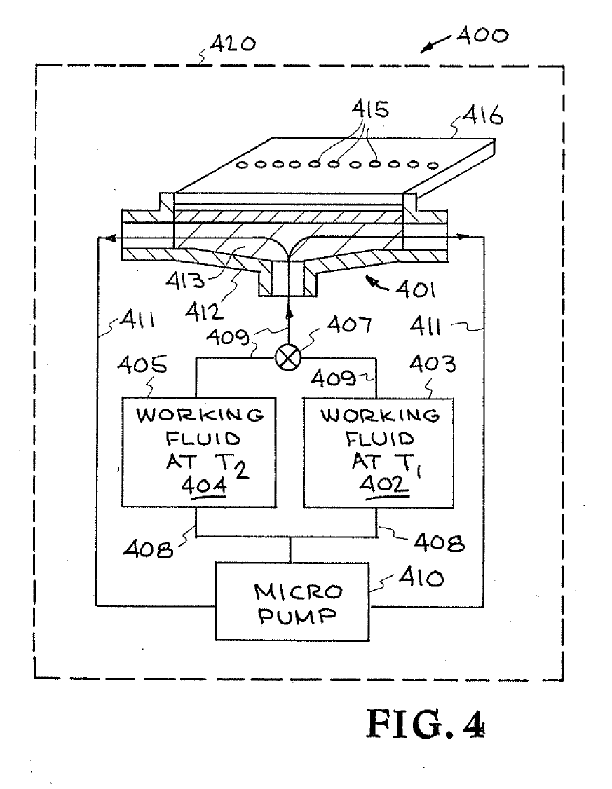

Referring now to FIG. 4, another embodiment of a thermal cycling system constructed in accordance with the present invention is illustrated. The system is designated generally by the reference numeral 400. The system 400 provides thermal cycling a material 415 to be thermal cycled between different temperatures using a microfluidic heat exchanger 401 operatively positioned with respect to the material 415 to be thermal cycled. The system 400 is contained in a compact, portable microfluidic-compatible platform 420.

The material 415 to be thermal cycled is contained on a microarray 416. Examples of microarrays are shown in U.S. Pat. No. 7,354,389 for a microarray detector and methods which states, “The present invention is directed to an analytic system for detection of a plurality of analytes that are bound to a biochip, wherein an optical detector uses registration markers illuminated by a first light source to determine a focal position for detection of the analytes that are illuminated by a second light source.” U.S. Pat. No. ______ for a microarray detector and methods is incorporated herein by reference.

The system 400 includes the following additional structural components: microfluidic heat exchanger housing 412, porous medium 413, micropump 410, lines 411, chamber 403, working fluid 402 at T₁, chamber 405, working fluid 404 at T₁, lines 408, multi-position valve 407, and lines 409. The structural components of the system 400 having been described, the operation of the system 400 will be explained. The multi-position valve 407 is actuated to provide flow of working fluid 402 at T₁ from chamber 403 to the microfluidic heat exchanger 401. Micro pump 410 is actuated driving working fluid 402 at T₁ from chamber 403 to the microfluidic heat exchanger 401. The working fluid 402 at T₁ passes through the porous medium 413 in the microfluidic heat exchanger 401 raising the temperature of the material to be thermalcycled 415 to temperature T₁. The porous medium 413 in the microfluidic heat exchanger 401 results in substantial surface area enhancement and increased fluid flow-path tortuosity, both of which enhance heat transfer and the resulting heat flux between the working fluid and the porous matrix.

Next the multi-position valve 407 is actuated to provide flow of working fluid 404 at T₂ from chamber 405 to the microfluidic heat exchanger 401. Micro pump 410 is actuated driving working fluid 404 at T₂ from chamber 405 to the microfluidic heat exchanger 401. The working fluid 402 at T₂ passes through the porous medium 413 in the microfluidic heat exchanger 401 lowering the temperature of the material to be thermalcycled 415 to temperature T₂.

The heat exchanger 401 of the system 400 utilizes inlet and exit channels where heating/cooling fluid 402 and 404 is passing through, an enclosure, and microarray 416 containing the material to be thermal cycled. The heat exchanger 401 is filled with a conductive porous medium 413 of uniform porosity and permeability. In another embodiment the enclosure is filled with a conductive porous medium 413 with a gradient porosity. The nominal permeability and porosity of the porous matrix are taken as 3.74×10⁻¹⁰ m² and 0.45, respectively. The porous medium 413 is saturated with heating/cooling fluid 402, 404 coming through an inlet channel. The inlet channel will be connected to hot and cold supply tanks 403 and 405. The switching multi-position valve 407 is used to switch between hot 402 and cold tanks 405 for heating and cooling cycles. All lateral walls and top of the porous medium are insulated to minimize losses. The micropump 410 is positioned to drive the working fluids 402 and 405 directly into the microfluidic heat exchanger 401. By positioning the micropump 410 outside the hot and cold supply tanks 403 and 405 and lines to the microfluidic heat exchanger 401 it eliminates the time the would be required to bring the micropump 410 up to the new temperature after each change.

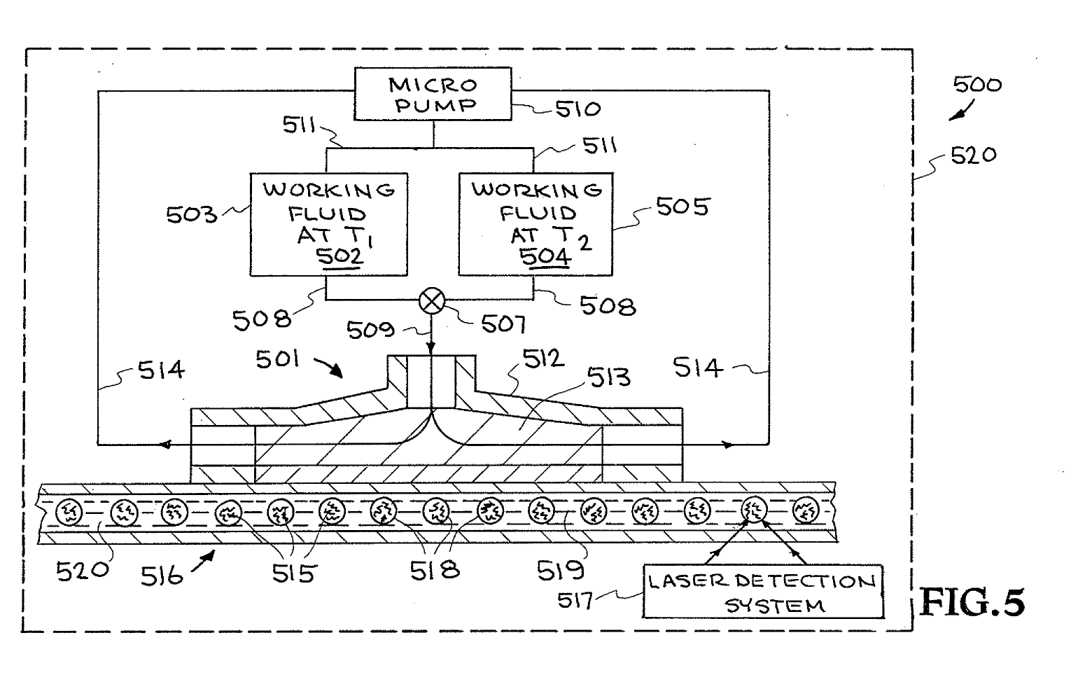

Referring now to the drawings and in particular to FIG. 5, one embodiment of a system constructed in accordance with the present invention is illustrated. The system is designated generally by the reference numeral 500. The system 500 provides extremely fast continuous flow or batch PCR amplification of target nucleic acids in a compact, portable microfluidic-compatible platform 520.

The system 500 provides thermal cycling a material 515 (DNA Sample) to be thermal cycled between a temperature T₁ and T₂ using a microfluidic heat exchanger 501 operatively positioned with respect to the material 515 to be thermal cycled. A working fluid 502 at T₁ is provided and the working fluid 502 at T₁ is flowed to the microfluidic heat exchanger 501. A working fluid 504 at T₂ is provided and the working fluid 504 at T₂ is flowed to the heat exchanger 501. The steps of flowing the working fluid at T₁ and at T₂ to the microfluidic heat exchanger 501 are repeated for a predetermined number of times. A porous medium 513 is located in the microfluidic heat exchanger 501. The working fluids at T₁ and T₂ flow through the porous medium 513 during the steps of flowing the working fluid at T₁ and T2 through the microfluidic heat exchanger 501. The system 500 is contained in a compact, portable microfluidic-compatible platform 520.

The material 515 to be thermal cycled is contained in droplets or microreactors 518. Systems for thermal cycling the droplets or microreactors 518 are described and illustrated in United States Published Patent No. 2008/0166793 by Neil Reginald Beer for sorting, amplification, detection, and identification of nucleic acid subsequences in a complex mixture. The disclosure of United States Published Patent No. 2008/0166793 by Neil Reginald Beer is incorporated herein by reference. The material 515 to be thermal cycled can for example be a DNA sample. The droplets or microreactors 518 are carried through a microchannel 520 in a chip 516 by a fluid 519. The material 515 (DNA sample) is analyzed by a laser detector system 517. The droplets or microreactors 518 are thermal cycled by the heat exchanger 501. the heat exchanger 501 provides microfluidic polymerase chain reaction (PCR) with extremely fast cycles, and a resulting extremely fast detection time for even long amplicons (amplified nucleic acids). The system 500 allows either singly or in combination: reagent and analyte mixing; cell, virion, or capsid lysing to release the target DNA if necessary; nucleic acid amplification through the polymerase chain reaction (PCR), and nucleic acid detection and characterization through optical or other means 517. An advantage of this system lies in its complete integration on a microfluidic platform and its extremely fast thermocycling.

The system 500 includes the following additional structural components: microfluidic heat exchanger housing 512, porous medium 513, micropump 510, lines 508, 509, 511, & 514, and multi position valve 507.

The structural components of the system 500 having been described, the operation of the system 500 will be explained. The valve 507 is actuated to provide flow of working fluid 502 at T₁ from chamber 503 to the microfluidic heat exchanger 501. Micro pump 510 is actuated driving working fluid 502 at T₁ from chamber 503 to the microfluidic heat exchanger 501. The working fluid 502 at T₁ passes through the porous medium 513 in the microfluidic heat exchanger 501 raising the temperature of the material to be thermalcycled 515 to temperature T₁. The porous medium 513 in the microfluidic heat exchanger 501 results in substantial surface area enhancement and increased fluid flow-path tortuosity, both of which enhance heat transfer and the resulting heat flux between the working fluid and the porous matrix.

Next the valve 507 is actuated to provide flow of working fluid 504 at T₂ from chamber 505 to the microfluidic heat exchanger 501. Micro pump 510 is actuated driving working fluid 504 at T₂ from chamber 505 to the microfluidic heat exchanger 501. The working fluid 502 at T₂ passes through the porous medium 513 in the microfluidic heat exchanger 501 lowering the temperature of the material to be thermalcycled 515 to temperature T₂. The steps of flowing the working fluid at T₁ and at T₂ to the microfluidic heat exchanger 501 are repeated for a predetermined number of times to provide the desired PCR. The porous medium 513 in the microfluidic heat exchanger 501 results in substantial surface area enhancement and increased fluid flow-path tortuosity, both of which enhance heat transfer and the resulting heat flux between the working fluid and the porous matrix.

The heat exchanger 501 of the system 500 utilizes inlet and exit channels where heating/cooling fluid 502 and 504 is passing through, an enclosure, and a layer of conductive plate attached to a PCR micro-chip. The enclosure is filled with a conductive porous medium 513 of uniform porosity and permeability. In another embodiment the enclosure is filled with a conductive porous medium 513 with a gradient porosity. The nominal permeability and porosity of the porous matrix are taken as 3.74×10⁻¹⁰ m² and 0.45, respectively. The porous medium 513 is saturated with heating/cooling fluid 502, 504 coming through an inlet channel. The inlet channel will be connected to hot and cold supply tanks 503 and 505. A switching valve 507 is used to switch between hot 502 and cold tanks 505 for heating and cooling cycles. All lateral walls and top of the porous medium are insulated to minimize losses. The micropump 510 is positioned to drive the working fluids 502 and 505 directly into the microfluidic heat exchanger 501. By positioning the micropump 510 outside the hot and cold supply tanks 503 and 505 and lines to the microfluidic heat exchanger 501 it eliminates the time the would be required to bring the micropump 510 up to the new temperature after each change.

Results

Tests and analysis were performed that provided unexpected and superior results and performance of apparatus and methods of the present invention. Some of the results and analysis of apparatus and methods of the present invention are described in the article “rapid microfluidic thermal cycler for polymerase chain reaction nucleic acid amplification,” by Shadi Mahjoob, Kambiz Vafai, and N. Reginald Beer in the International Journal of Heat and Mass Transfer 51 (2008) 2109-2122. The “Conclusions” section of the article states, “An innovative and comprehensive methodology for rapid thermal cycling utilizing porous inserts was presented for maintaining a uniform temperature within a PCR microchip consisting of all the pertinent layers. An optimized PCR design which is widely used in molecular biology is presented for accommodating rapid transient and steady cyclic thermal management applications. Compared to what is available in the literature, the presented PCR design has a considerably higher heating/cooling temperature ramps and lower required power while resulting in a very uniform temperature distribution at the substrate at each time step. A comprehensive investigation of various pertinent parameters on physical attributes of the PCR system was presented. All pertinent parameters were considered simultaneously leading to an optimized design.” The article “rapid microfluidic thermal cycler for polymerase chain reaction nucleic acid amplification,” by Shadi Mahjoob, Kambiz Vafai, and N. Reginald Beer in the International Journal of Heat and Mass Transfer 51 (2008) 2109-2122 is incorporated herein in it entirety by this reference.

The systems described above can include reprogrammable intermediate steps. The reprogrammable intermediate steps are described as follows and can be used with the systems described in connection with FIGS. 1-8:

A) With 2 tanks and the variable electronically-controlled valve, a thermal sensor upstream of the valve that is running under automated closed loop control provides the ability to adjust the ratios of the volume of flow from the T₁ and T₂ reservoirs. By adjusting these ratios ANY temperature between (and including) T₁ and T₂ are attainable. So say a thermal setpoint for T₃ is known by the user, they input T₁, T₂, & T₃ into their keypad, PC, pendant etc and the machine can thermal cycle between T₁ and T₂ and stop at T₃ if desired. For that matter, there can be multiple different “T₃”s as long as they are between T₁ and T₂.

B) This capability would be highly desirable for PCR since most protocols are 3-step, that is they cycle from the annealing (low) temperature (˜50 C) to an extension temperature (˜70 C) which is the temperature that the DNA polymerase enzyme performs optimally, to the high temperature (˜94 C) where the doubles strands separate. The sample is then brought back down to the anneal temp (˜50 C) and the cycle repeats. An example of the complete thermal cycling protocol, including one time reverse transcription (converts RNA to DNA) and enzyme activation (“hot start”) is given in the Experimental section (page 1855) of the publication “On-Chip Single-Copy Real-Time Reverse-Transcription PCR in Isolated Picoliter Droplets,” by N. Reginald Beer, Elizabeth K. Wheeler, Lorenna Lee-Houghton, Nicholas Watkins, Shanavaz Nasarabadi, Nicole Hebert, Patrick Leung, Don W. Arnold, Christopher G. Bailey, and Bill W. Colston in Analytical Chemistry Vol. 80, No. 6: Mar. 15, 2008 pages 1854-1858. The publication “On-Chip Single-Copy Real-Time Reverse-Transcription PCR in Isolated Picoliter Droplets,” by N. Reginald Beer, Elizabeth K. Wheeler, Lorenna Lee-Houghton, Nicholas Watkins, Shanavaz Nasarabadi, Nicole Hebert, Patrick Leung, Don W. Arnold, Christopher G. Bailey, and Bill W. Colston in Analytical Chemistry Vol. 80, No. 6: Mar. 15, 2008 pages 1854-1858 is incorporated herein by reference.

C) This capability also provides the ability for powering small molecule amplification that has multiple temperature steps that repeat in cycles. As time goes on, more and more of these molecular amplifications (not necessarily using DNA) will enter the art.

D) This also may be useful in other general chemical or complex synthesis reactions where endothermal and exothermal steps are required, such that an array or multi-well plate attached to this thermal cycler receives new reagents pipetted in (robotically or manually) at different temperatures in the repeating cycle.

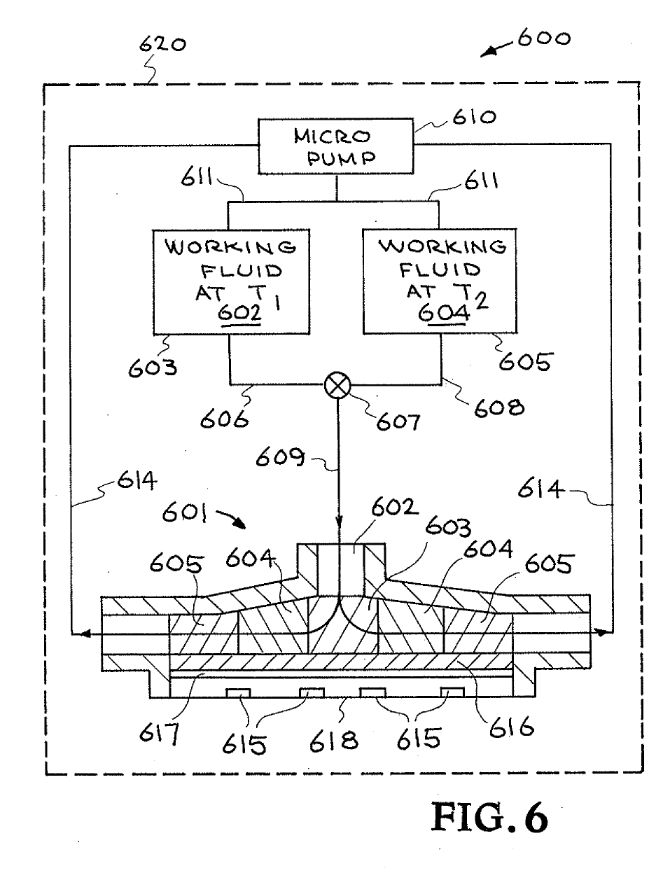

Referring now to FIG. 6, another embodiment of a thermal cycling system constructed in accordance with the present invention is illustrated. The system is designated generally by the reference numeral 600. The system 600 provides thermal cycling of a material to be thermal cycled between a temperature T₁ and T₂ using a microfluidic heat exchanger 601 operatively positioned with respect to the material 606 to be thermal cycled. The material to be thermal cycled is positioned in contact with the microfluidic heat exchanger 601 as illustrated in the previous figures.

A working fluid at T₁ is provided and the working fluid at T₁ is flowed to the microfluidic heat exchanger 601 through the inlet 602. A working fluid at T₂ is provided and the working fluid at T₂ is flowed to the heat exchanger 601. The steps of flowing the working fluid at T₁ and at T₂ to the microfluidic heat exchanger 601 are repeated for a predetermined number of times. A porous medium is located in the microfluidic heat exchanger 601. The working fluids at T₁ and T₂ flow through the porous medium during the steps of flowing the working fluid at T₁ and T₂ through the microfluidic heat exchanger 601. The porous medium is a porous medium of gradient permeability and porosity. The porous medium is made up of a first porous medium 603, a second porous medium 604, and a third porous medium 605. The first porous medium 603, second porous medium 604, and third porous medium 605 have different permeability and porosity. The first porous medium 603, second porous medium 604, and third porous medium 605 are arrange to provide a gradient permeability and porosity.

The structural components of the system 600 having been described, the operation of the system 600 will be explained. A valve is actuated to provide flow of working fluid at T₁ from a chamber to the microfluidic heat exchanger 601. A micro pump is actuated driving working fluid at T₁ from chamber to the microfluidic heat exchanger 601. The working fluid at T₁ passes through the porous medium in the microfluidic heat exchanger 601 raising the temperature of the material to be thermalcycled to temperature T₁. The porous medium with gradient permeability and porosity 603, 604, 605 in the microfluidic heat exchanger 601 results in substantial surface area enhancement and increased fluid flow-path tortuosity, both of which enhance heat transfer and the resulting heat flux between the working fluid and the porous matrix.

Next a valve is actuated to provide flow of working fluid at T₂ from a chamber to the microfluidic heat exchanger 601. A micro pump is actuated driving working fluid at T₂ from chamber to the microfluidic heat exchanger 601. The working fluid at T₂ passes through the porous medium 602 in the microfluidic heat exchanger 601 lowering the temperature of the material to be thermalcycled to temperature T₂. The steps of flowing the working fluid at T₁ and at T₂ to the microfluidic heat exchanger 601 are repeated for a predetermined number of times to provide the desired PCR. The porous medium with gradient permeability and porosity 603, 604, 605 in the microfluidic heat exchanger 601 results in substantial surface area enhancement and increased fluid flow-path tortuosity, both of which enhance heat transfer and the resulting heat flux between the working fluid and the porous matrix.

The aqueous channel can be used to mix various assay components (i.e., analyte, oligonucleotides, primer, TaqMan probe, etc.) in preparation for amplification and detection. The channel geometry allows for dividing the sample into multiple aliquots for subsequent analysis serially or in parallel with multiple streams. Samples can be diluted in a continuous stream, partitioned into slugs, or emulsified into droplets. Furthermore, the nucleic acids may be in solution or hybridized to magnetic beads depending on the desired assay. The scalability of the architecture allows for multiple different reactions to be tested against aliquots from the same sample. Decontamination of the channels after a series of runs can easily be performed by flushing the channels with dilute solution of sodium hypochlorite, followed by deionized water.

The heat exchanger 601 of the system 600 utilizes inlet and exit channels where heating/cooling fluid is passing through, an enclosure, and a layer of conductive plate attached to a PCR micro-chip or microarray. The enclosure is filled with a conductive porous medium of gradient porosity and permeability. The porous medium is saturated with heating/cooling fluid coming through an inlet channel 602. The inlet channel will be connected to hot and cold supply tanks. A switching valve is used to switch between hot and cold tanks for heating and cooling cycles. All lateral walls and top of the porous medium are insulated to minimize losses.

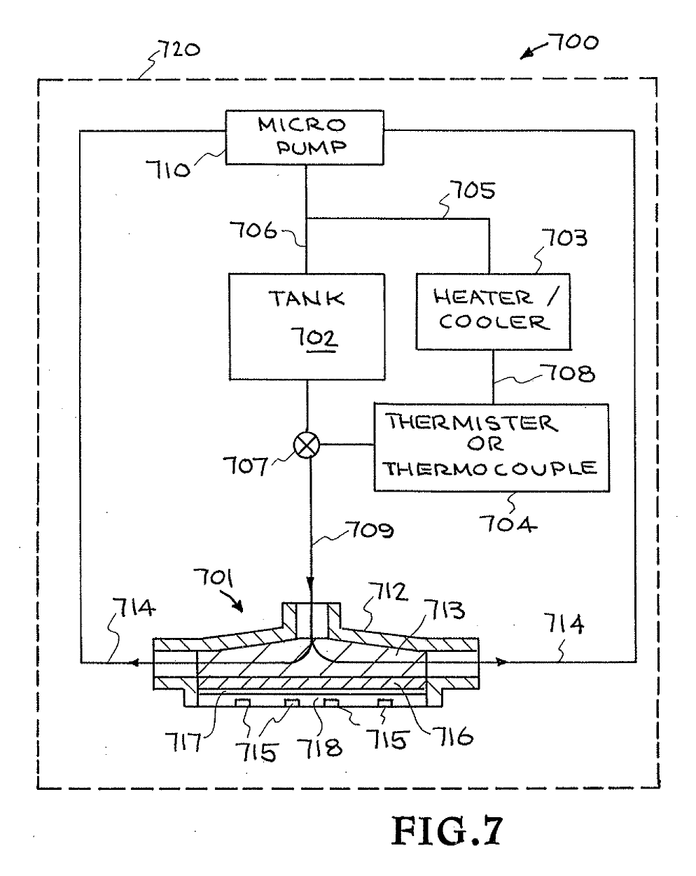

Referring now to the drawings and in particular to FIG. 7, another embodiment of a system constructed in accordance with the present invention utilizing a single tank is illustrated. The system is designated generally by the reference numeral 700. The system 700 provides extremely fast continuous flow or batch PCR amplification of target nucleic acids in a portable compact, portable microfluidic-compatible platform 720. The system 700 provides a 1-tank version where the single tank 702 is kept at a constant temperature and is fed by a return line(s) 714 and 706 from the heat exchanger 701. The same return line(s) 714 and 706 however feeds both the tank 702 as well as a separate tank bypass line 705. The bypass line 705 is essentially a coil with or without heatsinks and fans blowing over it that connects to the variable valve just upstream of the chip input. By placing a thermister or thermocouple 704 upstream of the variable valve 707, it is possible to send working fluid at T₁ or T₂ or any temperature in-between, and only requires 1 tank and heating system.

The material 715 to be thermal cycled is contained on a chip 718 containing the DNA. The DNA sample 715 is contained on the chip 718 containing the DNA sample. A highly conductive plate 716 connects the chip 718 to the heat exchanger 701. Conductive grease 717 is used to provide thermal conductivity between the chip 718 and the heat exchanger 701. Instead of conductive grease 717 between the chip 718 and the heat exchanger 701 other forms of connection may be used. For example, press-fit contact or thermally-conductive tape may be used between the chip 718 and the heat exchanger 701.

The system 700 provides thermal cycling a material 715 (DNA Sample) to be thermal cycled between a temperature T₁ and T₂ or any temperature in between using a microfluidic heat exchanger 701 operatively positioned with respect to the material 715 to be thermal cycled. The steps of repeatedly flowing the working fluid at T₁ and at T₂ to the microfluidic heat exchanger 701 provide PCR fast and efficient nucleic acid analysis. The microfluidic polymerase chain reaction (PCR) thermal cycling method 700 is capable of extremely fast cycles, and a resulting extremely fast detection time for even long amplicons (amplified nucleic acids). The method 700 allows either singly or in combination: reagent and analyte mixing; cell, virion, or capsid lysing to release the target DNA if necessary; nucleic acid amplification through the polymerase chain reaction (PCR), and nucleic acid detection and characterization through optical or other means. An advantage of this system lies in its complete integration on a microfluidic platform and its extremely fast thermocycling.

The system 700 includes the following structural components: microfluidic heat exchanger 701, microfluidic heat exchanger housing 712, porous medium 713, micropump 710, lines 705, 706, 708, 709, and 714, multi position valve 707, highly conductive plate 716, thermal grease 717, chip containing DNA sample 718, and DNA sample 715.

The structural components of the system 700 having been described, the operation of the system 700 will be explained. The valve 707 is actuated to provide flow of working fluid at T₁ from tank 702 to the microfluidic heat exchanger 701. The system 700 provides a 1-tank version where the single tank 702 is kept at a constant temperature and is fed by a return line(s) 714 and 706 from the heat exchanger 701. The same return line(s) 714 and 706 however feeds both the tank 702 as well as a separate tank bypass line 705. The bypass line 705 is essentially a coil with or without heatsinks and fans blowing over it that connects to the variable valve just upstream of the chip input. By placing a thermister or thermocouple 704 upstream of the variable valve 707, it is possible to send working fluid at T₁ or T₂ or any temperature in-between, and only requires 1 tank and heating system.

The porous medium 713 in the microfluidic heat exchanger 701 results in substantial surface area enhancement and increased fluid flow-path tortuosity, both of which enhance heat transfer and the resulting heat flux between the working fluid and the porous matrix. The heat exchanger 701 of the system 700 utilizes inlet and exit channels where heating/cooling fluid is passing through, an enclosure, and a layer of conductive plate attached to a PCR micro-chip. The enclosure is filled with a conductive porous medium 713 of uniform or gradient porosity and permeability. The porous medium 713 is saturated with heating/cooling fluid coming through an inlet channel. The switching valve 707 is used to switch between hot and cold for heating and cooling cycles. All lateral walls and top of the porous medium are insulated to minimize losses. The micropump 710 is positioned to drive the working fluids directly into the microfluidic heat exchanger 701. By positioning the micropump 710 outside the hot and cold supply tanks it eliminates the time that would be required to bring the micropump 710 up to the new temperature after each change.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

The invention claimed is:

1. A portable apparatus for thermal cycling a material to be thermal cycled, comprising:
a portable microfluidic-compatible platform,
a microfluidic heat exchanger carried by said portable microfluidic-compatible platform;
a porous medium in said microfluidic heat exchanger;
a microfluidic thermal cycling chamber containing the material to be thermal cycled, said microfluidic thermal cycling chamber operatively connected to said microfluidic heat exchanger;
a working fluid at first temperature
a first system for transmitting said working fluid at first temperature to said microfluidic heat exchanger;
a working fluid at a second temperature,
a second system for transmitting said working fluid at second temperature to said microfluidic heat exchanger;
a pump for flowing said working fluid at said first temperature from said first system to the microfluidic heat exchanger and through said porous medium;
and flowing said working fluid at said second temperature from said second system to said heat exchanger and through said porous medium.

2. The portable apparatus for thermal cycling of claim 1 wherein said first system for transmitting said working fluid at first temperature to said microfluidic heat exchanger is a first container for containing said working fluid at first temperature and said second system for transmitting said working fluid at second temperature to said microfluidic heat exchanger is a second container for containing said working fluid at second temperature.

3. The portable apparatus for thermal cycling of claim 1 wherein said first system for transmitting said working fluid at first temperature to said microfluidic heat exchanger and said second system for transmitting said working fluid at second temperature to said microfluidic heat exchanger comprises a single container and separate line with a heater or cooler that are connected to provide said working fluid at first temperature to said microfluidic heat exchanger and to provide said working fluid at second temperature to said microfluidic heat exchanger.

4. The portable apparatus for thermal cycling of claim 1 wherein said porous medium is a porous medium with uniform porosity or permeability.

5. The portable apparatus for thermal cycling of claim 1 wherein said porous medium is a porous medium with gradient porosity or permeability.

6. The portable apparatus for thermal cycling of claim 1 wherein the material to be thermal cycled is in a PCR chamber connected to said microfluidic heat exchanger.

7. The portable apparatus for thermal cycling of claim 1 wherein the material to be thermal cycled is in a multiwell plate connected to said microfluidic heat exchanger.

8. The portable apparatus for thermal cycling of claim 1 wherein the material to be thermal cycled is on a micro array connected to said microfluidic heat exchanger.

9. The portable apparatus for thermal cycling of claim 1 wherein said working fluid at a first temperature is a liquid working fluid.

10. The portable apparatus for thermal cycling of claim 1 wherein said working fluid at first temperature is a gas working fluid.

11. The portable apparatus for thermal cycling of claim 1 wherein said working fluid at first temperature is a liquid metal working fluid.

12. The portable apparatus for thermal cycling of claim 1 wherein said working fluid at second is a liquid working fluid.

13. The portable apparatus for thermal cycling of claim 1 wherein said working fluid at second temperature is a liquid working fluid.

14. The portable apparatus for thermal cycling of claim 1 wherein said working fluid at second temperature is a liquid metal working fluid.

15. The portable apparatus for thermal cycling of claim 1 including a working fluid at third temperature and a third container for containing said working fluid at third temperature and wherein said pump flows said working fluid at said third temperature from said third container to said microfluidic heat exchanger and through said porous medium.

16. A portable apparatus for thermal cycling a material to be thermal cycled between a temperature T₁ and T₂, comprising:
a portable microfluidic-compatible platform,
a microfluidic heat exchanger carried by said a portable microfluidic-compatible platform;
a porous medium in said microfluidic heat exchanger;
a microfluidic thermal cycling chamber containing the material to be thermal cycled, said microfluidic thermal cycling chamber operatively connected to said microfluidic heat exchanger;
a working fluid at T₁;
a first system for transmitting said working fluid at T₁ to said microfluidic heat exchanger;
a working fluid at T₂,
a second system for transmitting said working fluid at T₂ to said microfluidic heat exchanger;
a pump for flowing said working fluid at T₁ from said first system to the microfluidic heat exchanger and flowing said working fluid at T₂ from said second system to said heat exchanger and through said porous medium.

17. The portable apparatus for thermal cycling of claim 16 wherein said first system for transmitting said working fluid at T₁ to said microfluidic heat exchanger is a first container for containing said working fluid at first temperature and said second system for transmitting said working fluid at T₂ to said microfluidic heat exchanger is a second container for containing said working fluid at second temperature.

18. The portable apparatus for thermal cycling of claim 16 wherein said first system for transmitting said working fluid at T₁ to said microfluidic heat exchanger and said second system for transmitting said working fluid at T₂ to said microfluidic heat exchanger comprises a single container and separate line with a heater or cooler that are connected to provide said working fluid at T₁ to said microfluidic heat exchanger and to provide said working fluid at T₂ to said microfluidic heat exchanger.

19. The portable apparatus for thermal cycling of claim 16 wherein said porous medium is a porous medium with uniform porosity or permeability.

20. The portable apparatus for thermal cycling of claim 16 wherein said porous medium is a porous medium with gradient porosity or permeability.

21. A method of thermal cycling a material to be thermal cycled between a number of different temperatures, comprising the steps of:
providing a portable microfluidic-compatible platform,
providing a microfluidic heat exchanger on said portable microfluidic-compatible platform, said microfluidic heat exchanger operatively positioned with respect to the material to be thermal cycled
providing working fluid at a first temperature,
flowing said working fluid at said first temperature to the microfluidic heat exchanger to hold the material to be thermal cycled at said first temperature,
providing working fluid at a second temperature, and
flowing said working fluid at said first temperature to the heat exchanger to hold the material to be thermal cycled at said second temperature.

22. The method of thermal cycling of claim 21 including the step of providing a porous medium in the microfluidic heat exchanger and wherein said step of flowing said working fluid at said first temperature to the microfluidic heat exchanger comprises flowing said working fluid at said first temperature through said porous medium and wherein said step of flowing said working fluid at said second temperature to the microfluidic heat exchanger comprises flowing said working fluid at said second temperature through said porous medium.

23. The method of thermal cycling of claim 21 wherein said step of flowing said working fluid at said first temperature to the microfluidic heat exchanger and said step of flowing said working fluid at said second temperature to the microfluidic heat exchanger are repeated for a predetermined number of times.

24. The method of thermal cycling of claim 21 including the step of providing working fluid at a third temperature and flowing said working fluid at said third temperature the heat exchanger to cycle the material to hold the material to be thermal cycled at said third temperature.

25. A method of thermal cycling a material to be thermal cycled between a temperature T₁ and T₂ using a microfluidic heat exchanger operatively positioned with respect to the material to be thermal cycled, comprising the steps of:
providing working fluid at T₁,
flowing said working fluid at T₁ to the microfluidic heat exchanger,
providing working fluid at T₂, and
flowing said working fluid at T₂ to the heat exchanger.
